(19) United States
(12) Patent Application Publication
(10) Pub. No.: US 2012/0059380 A1
(43) Pub. Date: Mar. 8, 2012

(54) Bone Cement Delivery Systems and Related Kits and Methods

(75) Inventors: Terri Deangelo, Atkinson, NH (US); Jane Davis Golden, Hollis, NH (US); Alexandre Valentin, Belmont, MA (US)

(73) Assignee: WYETH LLC, Madison, NJ (US)

(21) Appl. No.: 13/256,006

(22) PCT Filed: Mar. 11, 2010
(86) PCT No.: PCT/US10/26919

§ 371 Date: Nov. 8, 2011

(30) Foreign Application Priority Data
Mar. 13, 2009 (US) 61160063

(51) Int. Cl. A61B 17/58

(52) U.S. Cl. 606/94

(57) ABSTRACT

A bone cement delivery method that includes inserting a portion of a bone cement delivery device into a femoral neck of a femur of a patient, and injecting bone cement paste into the femoral neck of the patient via the bone cement delivery system.

### TECHNICAL FIELD

This disclosure relates to bone cement delivery systems and related kits and methods.

### BACKGROUND

Bone cements, such as calcium phosphate based bone cements, can be used during certain medical treatments to help repair and/or reconstruct bone (e.g., fractured bone). The ability of certain bone cements to repair and/or reconstruct bone can be enhanced by the inclusion of recombinant human bone morphogenetic protein (rhBMP-2), which promotes the growth of bone. An example of a calcium phosphate based bone cement enhanced in this manner is rhBMP-2/CPM.

To prepare bone cements, such as calcium phosphate based bone cements, a powdery substance is generally combined with a liquid, and the resultant combination is mixed together to form a bone cement paste. The bone cement paste can then be delivered via a needle to a treatment site (e.g., a fracture site) to help repair and/or reconstruct the bone.

### SUMMARY

In one aspect of the invention, a bone cement delivery system includes an outer cannula including an elongate tubular member defining a bore extending from a proximal end of the elongate tubular member to a distal end of the elongate tubular member. The bone cement delivery system also includes a stylet configured to be removably positioned at least partially within the bore of the elongate tubular member. The bone cement delivery system is configured so that the outer cannula and the stylet can be percutaneously inserted into a femoral neck of a patient.

In another aspect of the invention, a bone cement mixing and delivery kit includes a bone cement mixing system and a bone cement delivery system. The bone cement delivery system includes an outer cannula including an elongate tubular member defining a bore extending from a proximal end of the elongate tubular member to a distal end of the elongate tubular member. The bone cement delivery system also includes a stylet configured to be removably positioned at least partially within the bore of the elongate tubular member. The bone cement delivery system is configured so that the outer cannula and the stylet can be percutaneously inserted into a femoral neck of a patient.

In an additional aspect of the invention, a bone cement delivery method includes inserting a portion of a bone cement delivery device into a femoral neck of a femur of a patient and injecting bone cement paste into the femoral neck of the patient via the bone cement delivery system.

Embodiments can include one or more of the following advantages.

In some embodiments, the elongate tubular member of the outer cannula has an outer diameter of about 0.094 inch to about 0.096 inch, and the elongate tubular member of the outer cannula has an inner diameter of about 0.075 inch to about 0.079 inch.

In some embodiments, the elongate tubular member of the outer cannula has a length of about six inches.

In some embodiments, the elongate tubular member is formed of stainless steel.

In some embodiments, the stylet is sized so that a distal end region of the stylet extends distally beyond the distal end of the elongate member.

In some embodiments, the distal end region of the stylet has a sharp tip.

In some embodiments, the bone cement delivery system further includes a handle attachable to the outer cannula.

In some embodiments, the bone cement mixing and delivery kit further includes a vial of lyophilized protein (e.g., recombinant human Bone Morphogenetic Protein-2 (rhBMP-2)) that can be reconstituted with sterile water. The resulting solution can then be injected into the bone cement mixing system to prepare a bone cement paste.

In some embodiments, the bone cement mixing and delivery kit further includes a syringe that can be used to withdraw the solution from the vial and then inject the solution into the bone cement mixing system.

In some embodiments, the bone cement mixing and delivery kit includes at least two stylets configured to be removably positioned at least partially within the bore of the elongate tubular member. One of the stylets has a sharp distal end and another of the stylets has a blunt distal end.

In some embodiments, the portion of the bone cement delivery system is percutaneously inserted into the femoral neck of the patient.

In some embodiments, the portion of the bone cement delivery device that is inserted into the femoral neck includes a distal end region of a cannula.

In some embodiments, the portion of the bone cement delivery device that is inserted into the femoral neck further includes a distal end region of a stylet, and the stylet is positioned at least partially within a bore of the cannula.

In some embodiments, a distal end of the stylet extends distally beyond a distal end of the cannula.

In some embodiments, the distal end of the stylet is sharp.

In some embodiments, the distal end of the stylet is blunt.

In some embodiments, inserting the portion of the bone cement delivery device into the femoral neck includes rotating the cannula and the stylet.

In some embodiments, the stylet includes a handle having at least one wall member configured to contact a handle of the cannula to substantially prevent the stylet from rotating relative to the cannula in at least a first rotational direction.

In some embodiments, injecting the bone cement paste into the femoral neck includes connecting a syringe to the cannula and operating the syringe to drive bone cement paste through a bore formed in the cannula.

In some embodiments, the method further includes removing a stylet from the bore formed in the cannula prior to connecting the syringe to the cannula.

In some embodiments, the bone cement delivery system comprises an outer cannula comprising an elongate tubular member defining a bore extending from a proximal end of the elongate tubular member to a distal end of the elongate tubular member, and a first stylet removably positioned at least partially within the bore of the elongate tubular member.

In some embodiments, the bone cement delivery method further includes, after passing a distal end of the first stylet through a cortex of the femur of the patient, removing the first stylet from the bore and inserting a second stylet into the bore.

In some embodiments, the distal end of the first stylet is sharp, and the distal end of the second stylet is blunt.

In some embodiments, the method further includes fixing the first stylet relative to the outer cannula.

In some embodiments, fixing the first stylet relative to the outer cannula includes axially fixing the first stylet relative to the outer cannula.

In some embodiments, fixing the first stylet relative to the outer cannula further includes fixing the first stylet relative to the outer cannula in at least one rotational direction.

In some embodiments, fixing the first stylet relative to the outer cannula includes rotating the first stylet relative to the outer cannula to position a projection extending from the outer cannula within a slot defined by the first stylet.

In some embodiments, the projection extends from a handle of the outer cannula, and the slot is defined by a head of the first stylet.

In some embodiments, fixing the first stylet relative to the outer cannula includes rotating the first stylet relative to the outer cannula to position projections of a locking member of the first stylet under members extending from a handle of the outer cannula.

In some embodiments, the locking member is a U-shaped member extending from a handle of the first stylet, and the projections extend laterally from opposing sides of the U-shaped member.

In some embodiments, the projections have contoured upper surfaces.

In some embodiments, the elongate tubular member of the outer cannula has an outer diameter of about 0.094 inch to about 0.096 inch, and the elongate tubular member of the outer cannula has an inner diameter of about 0.075 inch to about 0.079 inch.

In some embodiments, the elongate tubular member of the outer cannula has a length of about six inches.

In some embodiments, the bone cement paste includes calcium phosphate matrix (CPM) and recombinant human Bone Morphogenetic Protein-2 (rhBMP-2).

In some embodiments, the bone cement paste is injected into a portion of the femoral neck that is not fractured.

In some embodiments, the bone cement paste increases the bone mass of the femoral neck after a period of time.

Other aspects, features, and advantages will be apparent from the description and drawings, and from the claims.

### DESCRIPTION OF DRAWINGS

FIG. 1 is a perspective view of a disassembled bone cement delivery system.

FIG. 2 is a perspective view of the bone cement delivery system of FIG. 1 in an assembled configuration.

FIG. 3 is a perspective, top view of a sharp-tipped stylet of the bone cement delivery system of FIGS. 1 and 2.

FIG. 4 is a perspective, bottom view of the sharp-tipped stylet of the bone cement delivery system of FIGS. 1 and 2.

FIG. 5 is a perspective view of a blunt-tipped stylet that can be used interchangeably with the sharp-tipped stylet in the bone cement delivery system of FIGS. 1 and 2.

FIG. 6 is a perspective view of a bone cement mixing system.

FIG. 7 illustrates the bone cement delivery system of FIGS. 1 and 2 being introduced into the femur bone of a patient.

FIGS. 8A-8E are diagrammatical views of various stages of use of the bone cement delivery system of FIGS. 1 and 2 to inject bone cement paste into the femur of a patient.

FIG. 9 is a plan view of a bone cement mixing and delivery kit.

FIG. 10 is a perspective view of another disassembled bone cement delivery system.

FIG. 11 is a perspective view of the bone cement delivery system of FIG. 10 in an assembled configuration.

FIGS. 12 and 13 illustrate a process of securing a sharp-tipped stylet of the bone cement delivery system of FIGS. 10 and 11 to an outer cannula of the bone cement delivery system of FIGS. 10 and 11.

FIG. 14 is a perspective view of a blunt-tipped stylet that can be used interchangeably with the sharp-tipped stylet in the bone cement delivery system of FIGS. 10 and 11.

### DETAILED DESCRIPTION

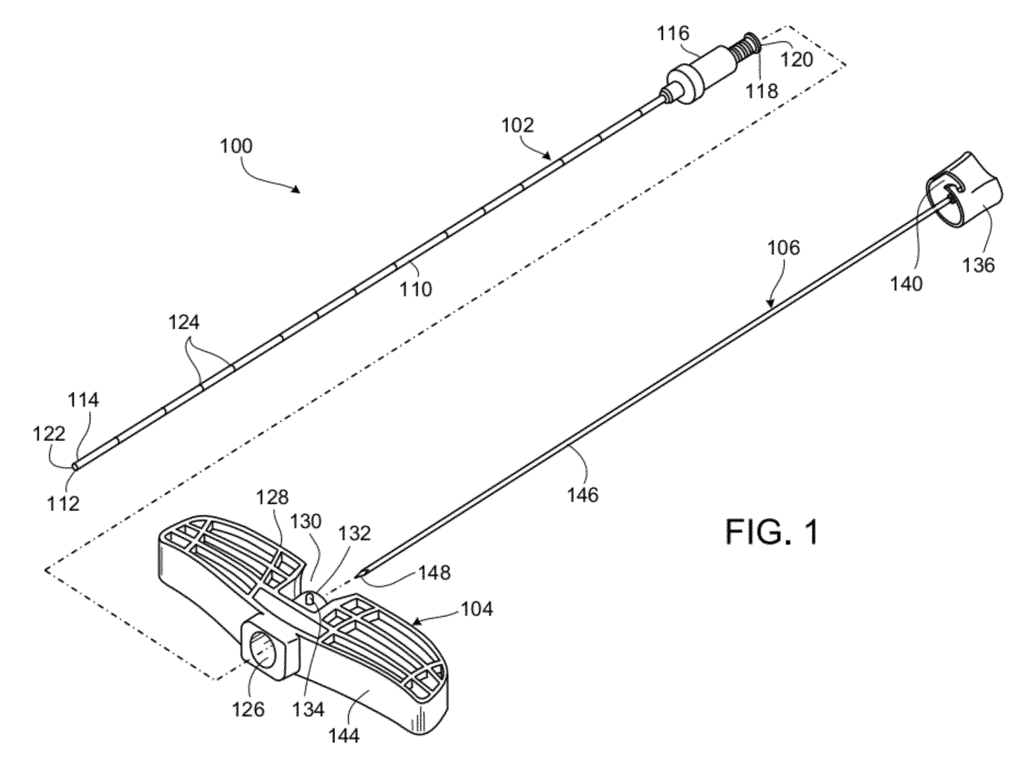

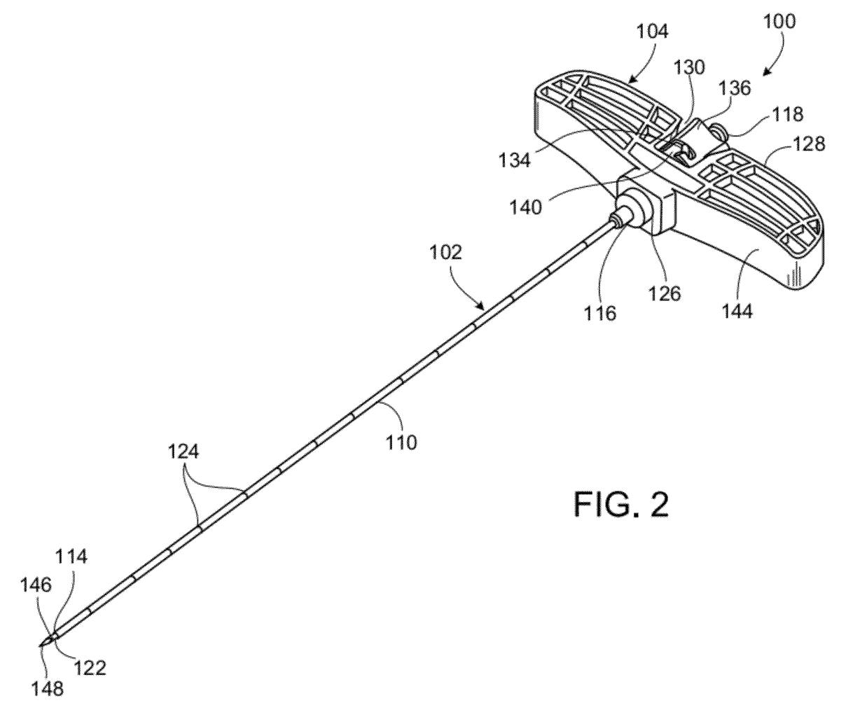

FIG. 1 is a perspective view of a bone cement delivery system 100 in a disassembled state, and FIG. 2 is a perspective view of the bone cement delivery system 100 in an assembled state. Referring to FIGS. 1 and 2, the bone cement delivery system 100 includes an outer cannula 102, a handle 104 that can be releasably attached to the outer cannula 102, and a sharp-tipped stylet 106 that fits within the outer cannula 102.

The outer cannula 102 includes a 13 gauge tubular member 110 formed of AISI 304 stainless steel. The outer diameter of the tubular member 110 is about 0.094 inch to about 0.096 inch, and the inner diameter is about 0.075 inch to about 0.079 inch. The tubular member 110 is about six inches in length. A bore 112 extends through the tubular member 110, from a proximal end to a distal end of the tubular member 110. The bore 112 has a diameter of about 0.075 inch to about 0.079 inch. A distal tip 114 of the tubular member 110 is tapered such that the wall thickness at the distal tip 114 of the decreases. This tapered arrangement can help to guide the distal tip 114 of the tubular member 110 through bone and other tissue during use.

Still referring to FIG. 1, the outer cannula 102 also includes a molded polymeric fitting 116 that is attached to a proximal end region of the tubular member 110. The molded fitting 116 can, for example, be molded onto the proximal end region of the tubular member 110 using insert molding techniques. Alternatively or additionally, the molded fitting 116 can be attached to the tubular member 110 using adhesive, thermal bonding, or mechanical fasteners. The proximal end region of the molded fitting 116 forms a male luer lock fitting 118 to which a syringe or other device including a corresponding female luer lock fitting can be attached. A bore 120 extends axially through the molded fitting 116 and aligns with the bore 112 of the tubular member 110 to form a central passage 122 that extends from the proximal end to the distal end of the outer cannula 102.

During use, the sharp-tipped stylet 106 can be disposed within the central passage 122 of the outer cannula 102, as shown in FIG. 2, to provide the tubular member 110 of the outer cannula 102 with increased rigidity. The dimensions of the tubular member 110 in combination with the relatively rigid material from which the tubular member 110 is formed, provide the tubular member 110 with sufficient rigidity and length, when used in combination with the stylet 106, to provide percutaneous access to the neck of a patient's femur (i.e., the femoral neck). The central passage 122 can also be used as a conduit for delivering material, such as bone cement paste, into the femur of a patient when the cannula 102 is disposed within the femur and the stylet 106 has been removed from the central passage 122 of the outer cannula 102.

The outer surface of the tubular member 110 of the outer cannula 102 is provided with axially spaced marker rings 124 that aid the user in positioning the tubular member 110 at a desired depth within the patient during use. As an alternative to or in addition to the axially spaced rings 124, markings of other types, such as numerical values, can be provided on the outer surface of the tubular member 110.

The handle 104, as shown in FIG. 1, includes a bore 126 that is sized and shaped to receive the molded fitting 116 of the outer cannula 102. The handle 104 is releasably attachable to the molded fitting 116 of the outer cannula 102. With the handle 104 attached to the molded fitting 116 of the outer cannula 102, the male luer lock fitting 118 at the proximal end of the molded fitting 116 extends sufficiently beyond a top surface 128 of the handle 104 to allow a syringe or other device including a corresponding female luer lock fitting to be secured to (i.e., screwed on to) the molded fitting 116.

The handle 104 includes a recessed region 130 that extends downward from the top surface 128 of the handle 104. A circular axial protrusion 132 extends upward from a surface of the handle 104 within the recessed region 130. A laterally extending locking pin or projection 134 extends from an outer surface of the axial protrusion 132. The recessed region 130 and the axial protrusion 132, as described in more detail below, are sized and shaped to receive and engage an enlarged head 136 of the stylet 106 when the stylet 106 is positioned within the central passage 122 of the outer cannula 102. The depth of the recessed region 130 can be substantially the same as the height of the head 136 of the stylet 106 such that a substantially continuous surface is provided across the top of the handle 104 when the stylet 106 is positioned in the central passage 122 of the outer cannula 102. With the head 136 of the stylet 106 positioned in the recessed region 130 of the handle 104, the laterally extending locking pin 134 can mate with a circumferentially extending L-shaped slot 140 formed in the head 136 of the stylet 106 to secure the stylet 106 to the handle/outer cannula assembly.

Referring to FIG. 2, the handle 104 is shaped so that the user can grasp the handle 104 with the user's palm contacting the top surface 128 of the handle 104 and the user's fingers wrapped around a bottom surface 144 of the handle 104. The outer cannula 102, which extends downward from the bottom surface 144 of the handle 104, can be positioned between two adjacent fingers (e.g., between the middle finger and the ring finger) of the user when the handle 104 is grasped in this manner. With the handle 104 and the outer cannula 102 grasped in this manner, the user can apply an axial and/or rotational force to the tubular member 110 of the outer cannula 102 via the handle 104 and molded fitting 116 to help drive the tubular member 110 through bone and other tissue.

In some embodiments, the handle 104 is formed of Acrylonitrile Butadiene Styrene (ABS). However, the handle 104 can be formed of other materials that provide a comfortable grasping surface for the user.

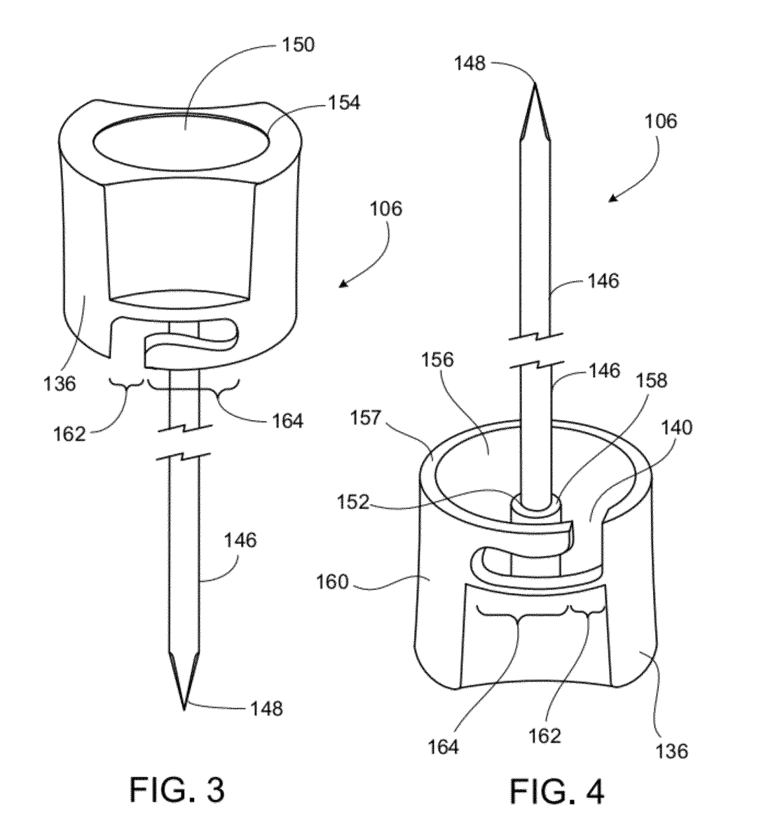

Referring to FIG. 3, which is a perspective view of the sharp-tipped stylet 106 from an upper side of the head 136, the sharp-tipped stylet 106 includes an elongate rod 146 with a sharp distal tip 148. A disk-shaped strike plate 150 is attached to the proximal end of the elongate rod 146. A circular recess 154 extends downward from a top surface of the head 136, and the strike plate 150 resides in the recess 154. The elongate rod 146 and the strike plate 150 are formed of AISI 304 stainless steel. The enlarged head 136 of the sharp-tipped stylet 106 is attached to the elongate rod 146 and the strike plate 150.

Referring to FIG. 4, which is a perspective view of the sharp-tipped stylet 106 from an underside of the head 136, the head 136 includes a bore 152 in which a proximal end region of the elongate rod 146 is disposed. An annular cavity 156 extends inward from a lower surface 157 of the head 136. The inner diameter of the annular cavity 156 is bound by an axial tubular projection 158 that extends downward from the top surface of the head 136 along the longitudinal axis of the head 136, and the outer diameter of the annular cavity 156 is bound by an outer wall 160 that extends downward from the periphery of the top surface of the head 136. The axial tubular projection 158 forms the bore 152 in which the elongate rod 146 is disposed.

The elongate rod 146 and the strike plate 150 can be attached to the head 136 using insert molding techniques. Alternatively or additionally, other attachment techniques, such as adhesive attachment and thermal bonding, can be used to attach the elongate rod and/or the strike plate to the head.

Still referring to FIG. 4, the L-shaped slot 140 of the head 136 is formed in the outer wall 160 and includes a vertical region 162 and a horizontal region 164. The L-shaped slot 140 is sized to receive and retain the laterally extending locking pin 134 of the handle 104. In particular, a central segment of the horizontal region 164 of the slot 140 has a width that is slightly less than the diameter of the locking pin 134 and an end segment of the horizontal region 164 of the slot 140 (i.e., the end segment of the horizontal region 164 that is opposite the vertical region 162 of the slot 140) has a width that is slightly greater than the locking pin 134.

With the vertical region 162 of the slot 140 of the stylet 106 aligned with the locking pin 134 of the handle 104, the stylet 106 can be loaded into the handle/outer cannula assembly such that the elongate rod 146 of the stylet 106 extends through the central passage 122 of the outer cannula 102 and the head 136 of the stylet 106 rests within the recessed region 130 of the handle 104. As the stylet 106 is loaded into the handle/outer cannula assembly the locking pin 134 of the handle 104 slides vertically through the vertical region 162 of the slot 140 and stops at the top of the vertical region 162. The stylet 106 can then be rotated so that the locking pin 134 slides within the horizontal region 164 of the slot 140, deflecting the outer wall 160 adjacent the central segment of the horizontal region 164 of the slot 140 before snapping securely into the wider end segment of the horizontal region 164 of the slot 140. In this configuration, the stylet 106 is inhibited (e.g., substantially prevented) from moving axially or rotationally relative to the handle/outer cannula assembly.

The outer diameter of the elongate rod 146 of the sharp-tipped stylet 106 is slightly smaller than the inner diameter of the outer cannula 102 (e.g., the inner diameter of the tubular member 110 of the outer cannula 102). Due to the size of the elongate rod 146 of the stylet 106 relative to the outer cannula 102, the stylet 106 can be passed through the central passage 122 of the outer cannula 102 and positioned within the central passage 122 of the outer cannula 102 with little lateral play. This arrangement helps to ensure that a sufficient amount of rigidity is provided to the tubular member 110 of the outer cannula 102 when the stylet 106 is positioned in the central passage 122 of the outer cannula 102. This arrangement also helps to ensure that no material passes up the central bore 122 when the stylet 106 and cannula 102 are inserted into bone and/or tissue of a patient.

The stylet 106 is sized so that the sharp distal tip 148 of its elongate rod 146 extends past the distal end of the outer cannula 102 when the stylet 106 is positioned in and axially and rotationally locked relative to the handle/outer cannula assembly, as shown in FIG. 2. Due to the position of the sharp tip 148 distal to the distal end of the cannula 102, the sharp tip 148 can facilitate passage of the outer cannula 102 into and through bone and other tissue.

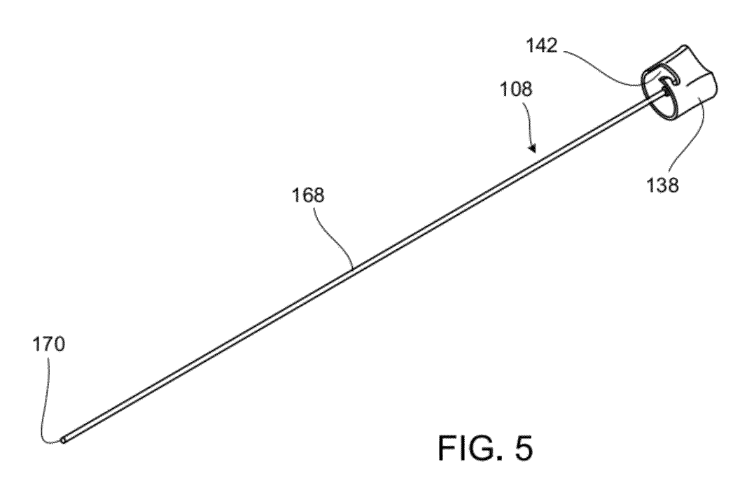

FIG. 5 shows a blunt-tipped stylet 108 that can be used interchangeably with the sharp-tipped stylet 106 in the bone cement delivery system 100. Unlike the sharp-tipped stylet 106, the blunt-tipped stylet 108 includes an elongate rod 168 with a blunt distal end 170. The blunt-tipped stylet 106 includes a head 138 with an L-shaped slot 142 that is substantially identical to the head 136 of the sharp-tipped stylet 106. All other features (e.g., size, shape, materials, construction) of the blunt-tipped stylet 108 are also generally the same as the sharp-tipped stylet 106. Therefore, those features will not be described in detail.

The above-described bone cement delivery system 100 has been found to work particularly well for delivering bone cement paste (i.e., a mixture of calcium phosphate matrix (CPM) and recombinant human bone morphogenetic protein (rhBMP-2)) into a femur (e.g., a femoral neck) of a patient. This type of bone cement paste has the ability to induce bone growth when administered in the bone of a patient.

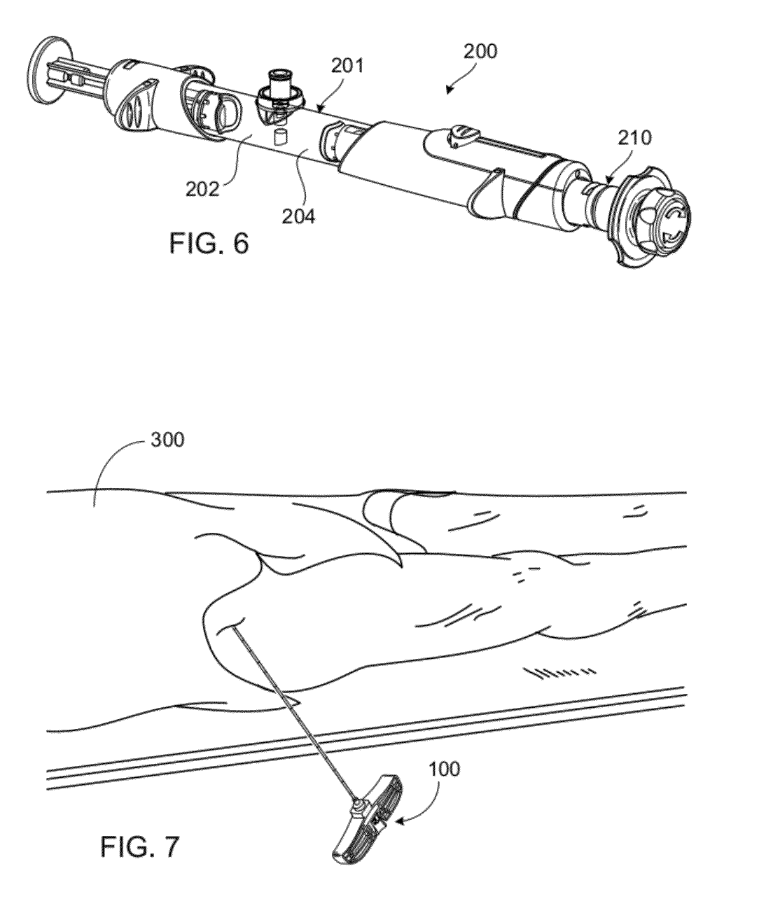

Prior to using the bone cement delivery system 100 to deliver bone cement paste into a patient, the bone cement paste is first prepared (e.g., mixed) in a bone cement mixing system. FIG. 6 is a perspective view of a bone cement mixing system 200 that can be used to prepare the bone cement paste. The bone cement mixing system 200 includes a housing 201 that forms two mixing chambers 202, 204. The mixing chambers 202, 204 initially contain a dry calcium phosphate/sodium bicarbonate powder. The bone cement mixing system 200 is configured so that liquid, such as liquid containing rhBMP-2, can be delivered into the mixing chambers 202, 204, and the combination of the liquid and the powder can be transferred back and forth between the mixing chambers 202, 204 to mix the liquid and powder together. After this first stage of mixing, the bone cement mixing system 200 can be reconfigured for a second stage of mixing during which the mixture of powder and liquid is passed back and forth between the mixing chamber 202 formed in the housing 201 and another mixing/delivery chamber formed in a bone cement delivery syringe 210 that is releasably secured to the housing 201. After the second stage of mixing, the liquid and powder mixture can be collected in the mixing/delivery chamber of the bone cement delivery syringe 210, and the bone cement delivery syringe 210 can be removed from the housing 201 of the bone cement mixing system 200. After removing the bone cement delivery syringe 210 from the rest of the bone cement mixing system 200, the bone cement delivery syringe 210 can be used to deliver the bone cement paste through the central passage 122 of the outer cannula 102 of the bone cement delivery system 100 and into the femur of a patient, as described in more detail below. The bone cement mixing system 200 is described in greater detail in U.S. Patent Application Publication No. 2008-0065088, which is incorporated by reference herein.

Referring to FIG. 7, a method of injecting bone cement paste into the proximal femur of a patient 300 is typically carried out with the patient 300 supine on a table that can accommodate fluoroscopy. The bone cement delivery system 100 with the sharp tipped stylet 106 disposed within and extending from the central passage 122 of the outer cannula 102 is introduced through the patient's skin and driven toward the patient's femur in an area that extends approximately two centimeters inferior to the base of the greater trochanter of the patient's femur. In order to locate the region that is approximately two centimeters inferior to the base of the greater trochanter, the patient's leg can be placed in a lateral recumbent position and external anatomical landmarks (e.g., the greater trochanter and proximal femoral shaft) can be manually palpated. In addition to these external landmarks, fluoroscopic imaging can be used to ensure that the bone cement delivery system 100 is introduced into the desired region of the patient's leg.

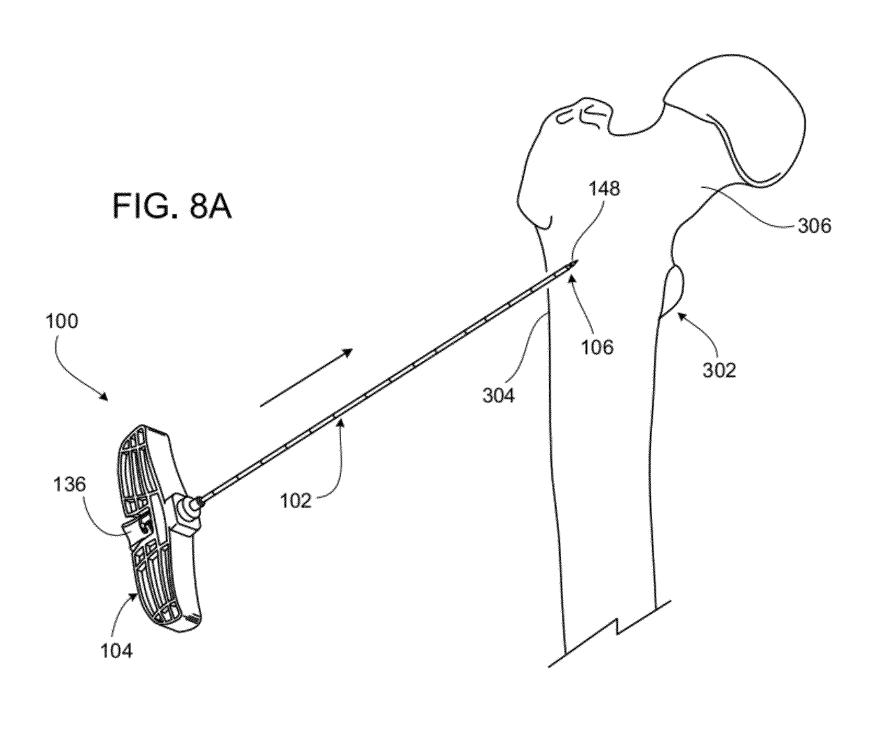

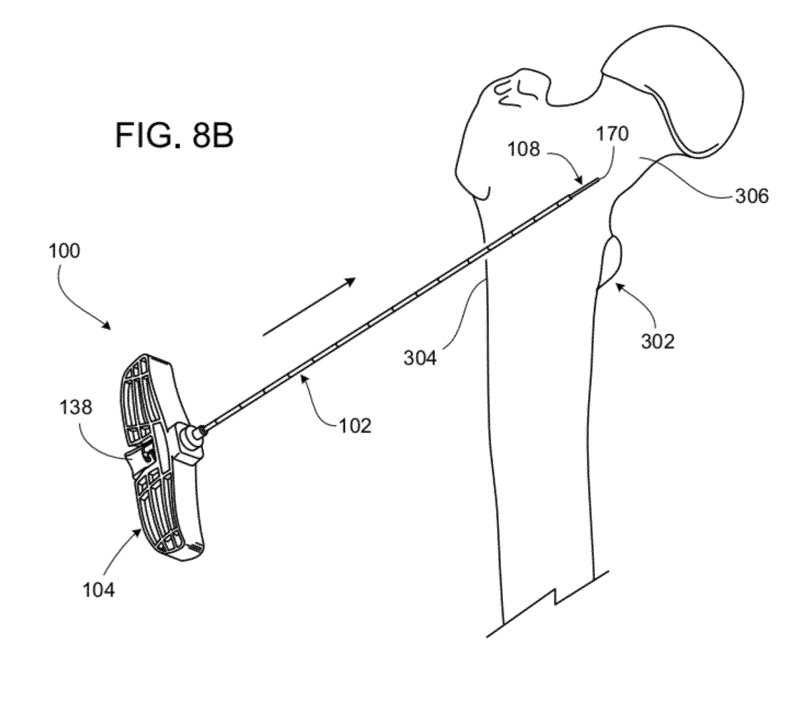

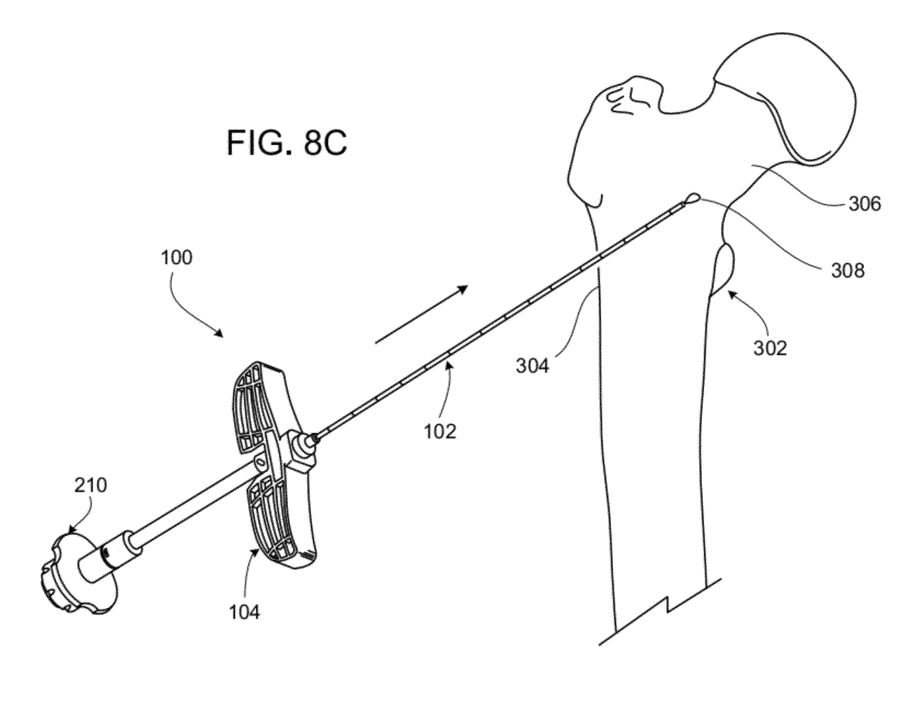

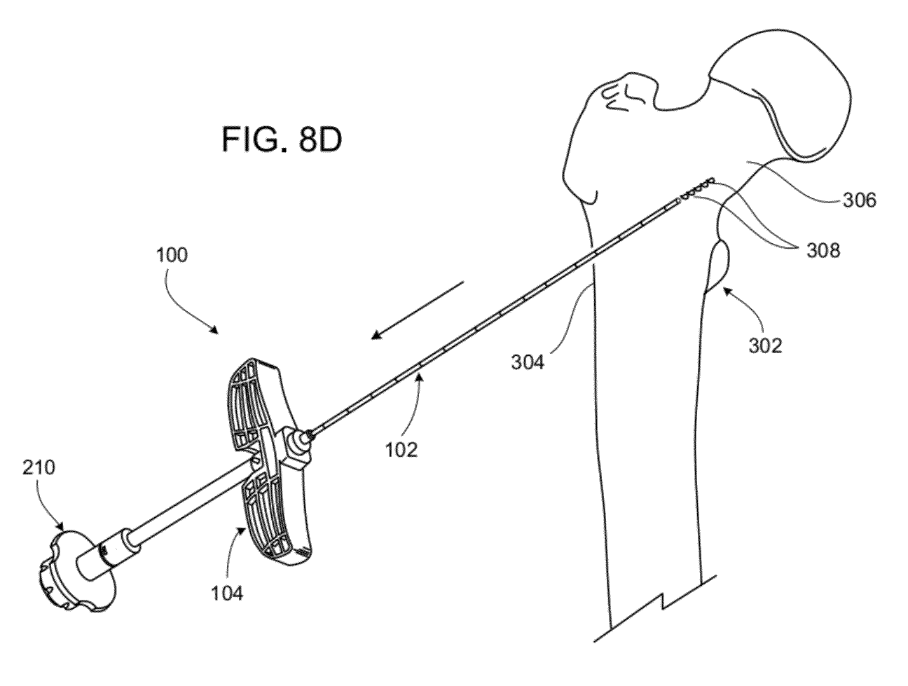

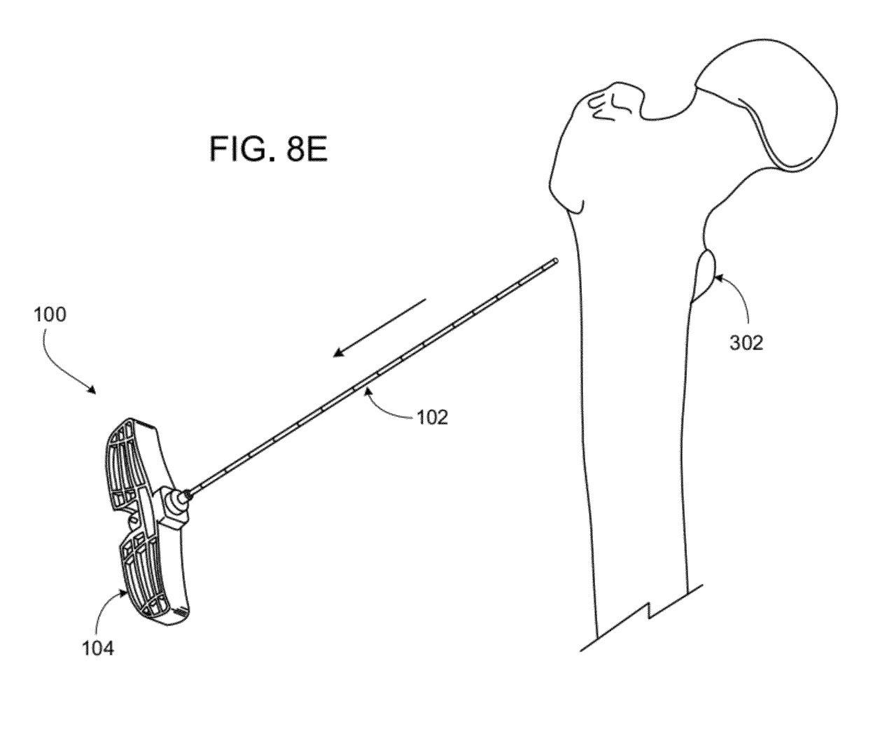

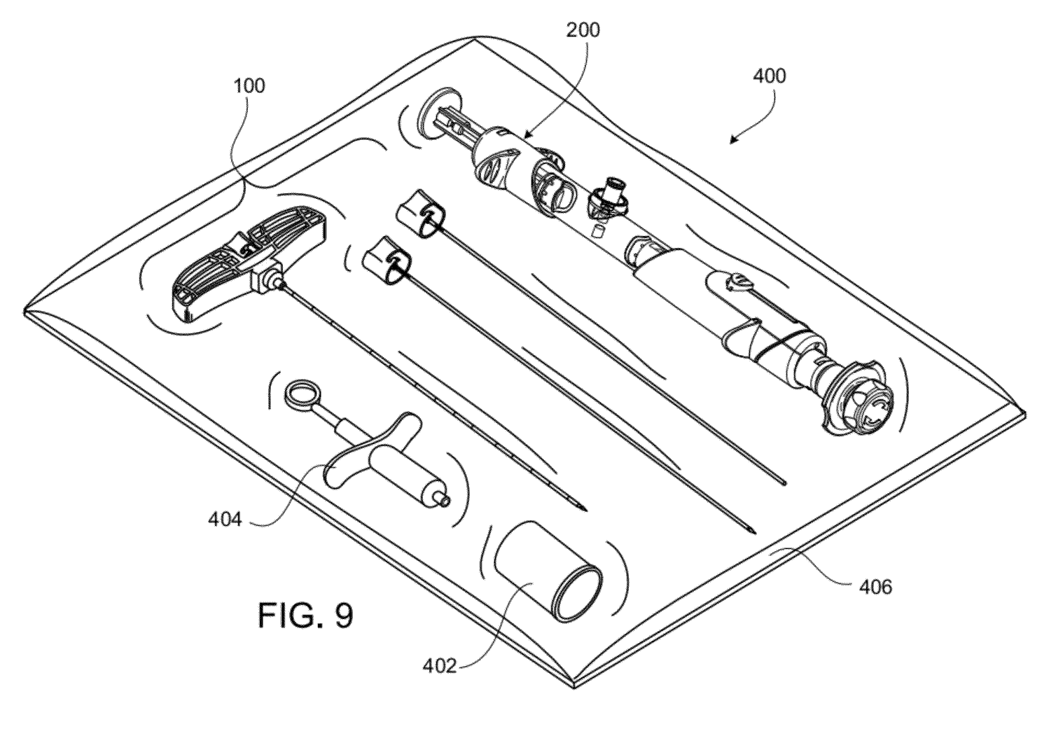

FIGS. 8A-8E are diagrammatical views of various stages of use of the bone cement delivery system 100 to inject bone cement paste into the femur 302 of the patient 300. Referring to FIG. 8A, after the outer cannula 102 and the sharp-tipped stylet 106 have been passed through the skin, fat, and muscle of the patient and brought into contact with the hard bony cortex 304 of the femur 302, the position of the sharp tip 148 of the stylet 106 is again viewed fluoroscopically to ensure that the portion of the bone cement delivery system 100 within the patient 300 is positioned as desired (e.g., in contact with the desired region of the femur 302 and at a desired angle). After confirming that the bone cement delivery system 100 is positioned as desired, the surgeon manually rotates the stylet 106 in a clock-wise fashion while applying an axial force to the outer cannula 102 and the sharp-tipped stylet 106 via the handle 104 to drive the sharp tip 148 of the stylet 106 through the cortex 304 and toward the femoral neck 306 of the patient's femur 302. As an alternative to or in addition to manually advancing the outer cannula 102 and the stylet 106 through the cortex 304, the user can strike the strike plate 150 of the stylet 106 with a mallet until the sharp tip 148 of the stylet 106 passes through the cortex 304.

After the sharp tip 148 of the stylet 106 has passed through the cortex 304 of the femur 302, the sharp-tipped stylet 106 is removed from the central passage 122 of the outer cannula 102 and replaced with the blunt-tipped stylet 108. To remove the sharp-tipped stylet 106 from the central passage 122 of the outer cannula 102, the user grasps the head 136 of the stylet 106 and rotates the stylet 106 until the locking pin 134 extending from the handle 104 is positioned within the vertical region 162 of the L-shaped slot 140 formed in the head 136 of the stylet 106. The sharp-tipped stylet 106 is then pulled out of the central passage 122 of the outer cannula 102. After removing the sharp-tipped stylet 106 from the outer cannula 102, the blunt-tipped stylet 108 is inserted into the central passage 122 of the outer cannula 102. Once the blunt-tipped stylet 108 has been fully inserted into the central passage 122 of the outer cannula 102, the user rotates the head 138 of the stylet 108 until the locking pin 134 of the handle 104 becomes disposed within the end segment of the horizontal region of the L-shaped slot 142 formed in the head 138 of the blunt-tipped stylet 108 to axially fix the stylet 108 relative to the handle 104 and the outer cannula 102.

Referring to FIG. 8B, with the blunt-tipped stylet 108 securely positioned within the outer cannula 102, the outer cannula 102 and the stylet 108 are manually advanced (by applying an axial and rotational force to the outer cannula 102 and the stylet 108 via the handle 104) to the point where the femoral shaft aligns centrally with the greater trochanter of the femur 302. The blunt-tipped stylet 108 experiences more resistance when passing through the bone than the sharp-tipped stylet 106. Thus, it has been found that replacing the sharp-tipped stylet 106 with the blunt-tipped stylet 108 after passing through the hard cortex 304 of the femur 302 substantially reduces the likelihood of inadvertently driving the system all the way through the femur. After verifying that the bone cement delivery system 100 is positioned within a central region of the femur 302 and at a desired angle by fluoroscopically viewing the portion of the bone cement delivery system 100 within the patient's femur 302, the outer cannula 102 and the stylet 108 are further advanced until the blunt tip 170 of the stylet 108 and the distal end of the outer cannula 102 are positioned at the base of the femoral neck. This positioning of the system is once again fluoroscopically verified.

As an alternative to or in addition to manually advancing the outer cannula 102 and the blunt-tipped stylet 108 within the femur 302, the user can gently strike the strike plate of the stylet 108 with a mallet until the blunt tip 170 of the stylet 108 is positioned at a desired location within the femur 302.

After fluoroscopically verifying that the blunt tip 170 of the stylet 108 and the distal end of the outer cannula 102 are positioned as desired within the femoral neck 306, the user detaches the blunt-tipped stylet 108 from the handle 104 and removes the blunt-tipped stylet 108 from the outer cannula 102. As a result, only the outer cannula 102 of the bone cement delivery system 100 remains within the femur 302. A syringe containing approximately one milliliter of saline or water is then secured to the male luer lock fitting 118 extending from the outer cannula 102 near the top of the handle 104 and the saline or water is injected through the central passage 122 of the outer cannula 102 and into the femur 302 to clear the outer cannula 102 of any obstructions. After flushing the central passage 122 of the outer cannula 102, the syringe is removed from the handle 104.

Referring to FIG. 8C, the bone cement delivery syringe 210 of the bone cement mixing system 200 is then secured to the outer cannula 102 by screwing a female luer lock fitting on the distal end of the bone cement delivery syringe 210 onto the male luer lock fitting 118 extending from the proximal end of the outer cannula 102. As a result, a delivery chamber of the bone cement delivery syringe 210, which contains rhBMP-2/CPM bone cement paste (i.e., bone cement paste formed of injectable calcium phosphate matrix (CPM) and recombinant human Bone Morphogenetic Protein-2 (rhBMP-2)), is placed in fluid communication with the central passage 122 of the outer cannula 102. With the bone cement delivery syringe 210 secured to outer cannula 102, a consistent pressure is applied to the plunger (or piston) of the bone cement delivery syringe 210 to slowly inject a bolus 308 of the bone cement paste into the femoral neck 306.

As shown in FIG. 8D, multiple boluses 308 (approximately one milliliter each) of the bone cement paste are injected into the femoral neck 306. The outer cannula 102 is retracted slightly prior to the injection of each bolus 308 so that the bone cement paste is distributed throughout the intertrochanteric region of the femur 302. For example, the outer cannula 102 can be refracted by about one millimeter to two millimeters prior to each bolus injection. In some embodiments, a total of about three milliliters to about eight milliliters (e.g., about three milliliters to about six milliliters, about three milliliters to about five milliliters, about four milliliters to about six milliliters, about six milliliters) of the bone cement paste is injected into the femur 302 during treatment.

The recombinant human Bone Morphogenetic Protein-2 (rhBMP-2) is an osteoinductive protein that directs uncommitted mesenchymal cells to differentiate into osteoblasts. As combined with the injectable calcium phosphate matrix (CPM), the rhBMP-2 can be delivered locally into the femur 302, as described above, to induce bone growth within the femur 302. The femur 302 into which the bone cement paste is injected is typically at risk of fracture due to osteoporosis or other bone weakening conditions. However, unlike certain conventional treatments, the bone cement paste is being injected into a region of the bone that is not yet fractured. By doing this, bone mass at the site of increased fracture risk can be increased to reduce the likelihood of fracture.

Referring to FIG. 8E, after a desired amount of bone cement paste has been injected into the femur 302, the bone cement delivery syringe 210 is detached from the bone cement delivery system 100, and the outer cannula 102 is removed from the patient.

In some cases, prior to removing the outer cannula 102 from the femur 302 of the patient 300, the blunt-tipped stylet 108 is reinserted into the central passage 122 of the outer cannula 102 to eject any remaining bone cement paste from the cannula 102. In such cases, after ejecting the bone cement paste, the outer cannula 102 and blunt-tipped stylet 108 can together be removed from the patient.

FIG. 9 illustrates a bone cement mixing and delivery kit 400. The kit 400 includes the bone cement delivery system 100, the bone cement mixing system 200, a vial 402 of lyophilized rhBMP-2 402, and a syringe 404. The various components of the bone cement mixing and delivery kit 400 are contained in a single package (e.g., a plastic tray-like package) 406 that can be provided to the user in a sterilized condition (e.g., in a sterile bag).

While certain embodiments have been described above, other embodiments are possible.

While the tubular member 110 of the outer cannula 102 has been described as being formed of stainless steel, other materials that provide the tubular member 110, when used in combination with one of the stylets 106, 108, with sufficient rigidity to percutaneously access the femur (e.g., the femoral neck) of a patient can be used.

While the molding fitting 116 of the outer cannula 102 has been described as being formed of one or more polymeric materials, the molded fitting 116 can alternatively or additionally be formed of one or more other materials, such as metal.

While the molding fitting 116 of the outer cannula 102 has been described as being a component that is molded separately from the tubular member 110 of the outer cannula 102 and then attached to the tubular member 110, the fitting 116 can alternatively be integrally formed with the tubular member 110.

While the molding fitting 116 of the outer cannula 102 has been described as including a male luer lock fitting that allows syringes and other devices including corresponding female luer lock fittings to be attached to the outer cannula 102, the molded fitting of the outer cannula can alternatively or additionally include other types of fittings that allow syringes or other devices to be secured to the outer cannula.

While the handle 104 has been described as being releasably attached to the molded fitting 116 of the outer cannula 102, the handle 104 can alternatively be permanently attached (e.g., adhesively attached or thermally bonded) to the molded fitting 116.

While the handle 104 has been described as being a component that is molded separately from the molding fitting 116 of the outer cannula 102 and then attached to the molded fitting 116, the handle 104 can alternatively be integrally formed with the molded fitting 116.

While the strike plates have been described as being attached to the elongate rods 146, 168 of the sharp-tipped and blunt-tipped stylets 106, 108, the strike plate and the elongate rod of each stylet can alternatively be fabricated form a unitary piece of metal.

While the elongate rods 146, 148 and strike plates of the sharp-tipped and blunt-tipped stylets 106, 108 have been described as being formed of stainless steel, they can alternatively or additionally be formed of one or more other metals or alloys.

While the heads 136, 138 of the sharp-tipped stylet 106 and the blunt-tipped stylet 108 have been described as being formed of one or more polymeric materials, the heads 136, 138 can alternatively or additionally be formed of one or more other materials, such as metal.

While the stylets 106, 108 have been described as being secured to the handle 104 during use by snapping the locking pin 134 of the handle 104 within the L-shaped slots 140, 142 of the stylets 106, 108, other types of locking arrangements can be used. In some embodiments, for example, the stylets are provided with locking pins that cooperate with slots formed in the handle to secure the stylets to the handle.

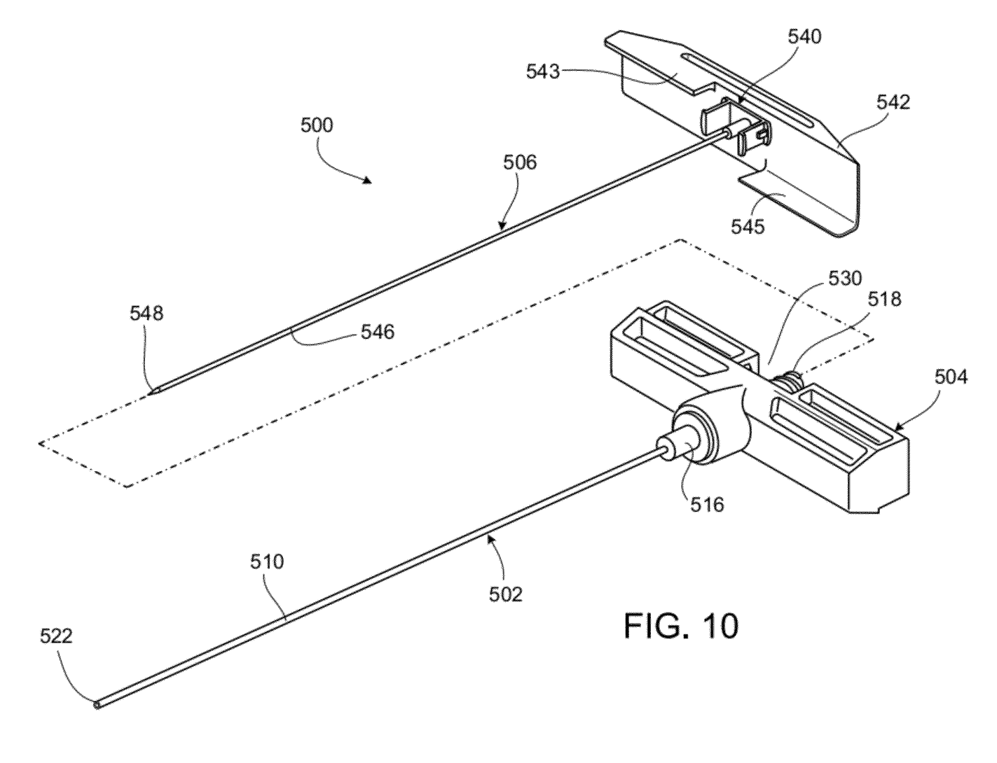

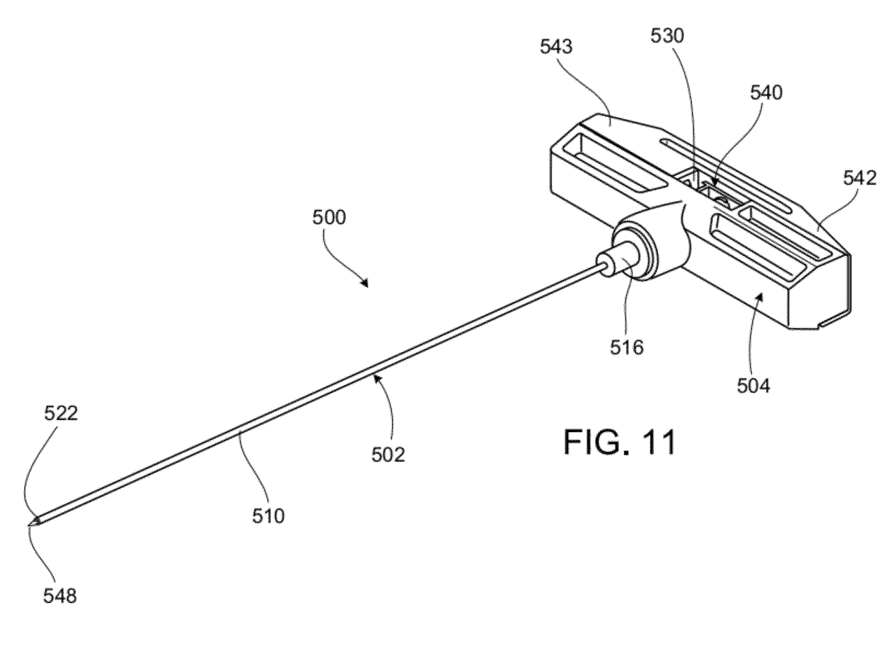

FIGS. 10 and 11 illustrate another example of a bone cement delivery system 500 including an outer cannula 502 and a sharp-tipped stylet 506 that fits within the outer cannula 502 and can be releasably secured to the outer cannula 502. FIG. 10 shows the bone cement delivery system 500 in a disassembled state, and FIG. 11 shows the bone cement delivery system 500 in an assembled state. The outer cannula 502 includes a tubular member 510 and a molded fitting 516 attached to an end region of the tubular member 510. A handle 504 is attached (e.g., thermally bonded or adhesively bonded) to the molded fitting 516 of the outer cannula 502. Bores extend axially through the handle 504, the tubular member 510, and the molded fitting 516 to form a central passage 522 that extends from the proximal end to the distal end of the outer cannula 502. The outer cannel 502 can have any of the dimensions and can be formed of any of the materials described herein with respect to the outer cannula 102. The handle 504 can be formed of any of the materials described herein with respect to the handle 104.

As shown in FIG. 10, the handle 504 defines a recessed region 530 sized and shaped to receive a locking mechanism 540 extending from a handle 542 of the sharp-tipped stylet 506 when the stylet 506 is mated with the outer cannula 502 in the manner shown in FIG. 11. A luer lock fitting 518 extends upward from a surface of the handle 504 within the recessed region 530. When the stylet 506 is removed from the outer cannula 502, as shown in FIG. 10, a syringe can be connected to the luer lock fitting 518 and used to inject material (e.g., saline, water, bone cement paste, etc.) through the central passage 522 of the outer cannula 502.

Still referring to FIG. 10, the sharp-tipped stylet 506 includes an elongate rod 546 with a sharp distal tip 548. The handle 542 of the stylet 506 is attached to the proximal end region of the elongate rod 546. The handle 542 includes wall segments 543, 545 that are sized and shaped to abut a portion of the handle 504 of the outer cannula 102 when the stylet 506 and the outer cannula 502 are mated. The wall segments 543, 545 help prevent the stylet 506 from rotating relative to the handle 504 of the outer cannula 502 past a desired amount. The wall segments 543, 545 can, for example, prevent the stylet 506 from rotating more than 180 degrees relative to the handle 504 of the outer cannula 502 when the stylet 506 is fully inserted into the central passage 522 of the outer cannula 502. The wall segments 543, 545 can be used to transmit rotational forces (e.g., rotational forces applied by the user) from the handle 542 of the stylet 506 to the handle 504 of the outer cannula 502.

In some embodiments, a strike plate is attached to or integrally formed with the elongate rod 546 and exposed along the top surface of the handle 542. The strike plate can be formed of any of the materials discussed above with respect to the strike plate 150 and can be attached to the elongate rod 546 using any of the various techniques described above for attaching the strike plate 150 to the elongate rod 146. During treatment, the surgeon can strike the strike plate with a mallet during a surgical procedure to transmit forces along the elongate rod 546 and the outer cannula 502, helping to drive the sharp tip 548 of the elongate rod 546 and the outer cannula 502 through bone and tissue.

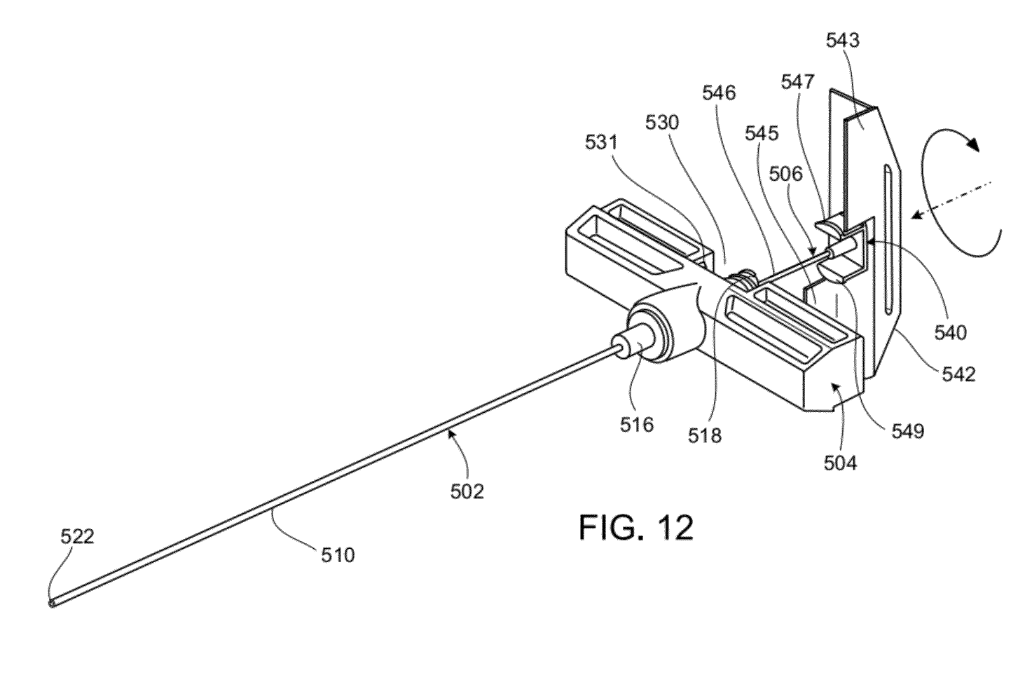

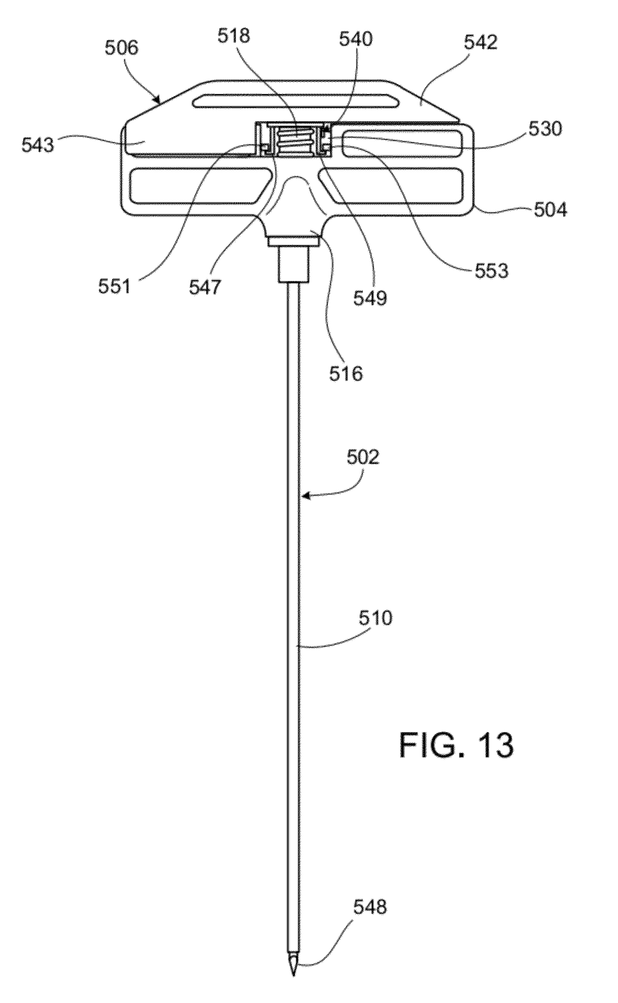

FIGS. 12 and 13 illustrate a process of securing the sharp-tipped stylet 506 to the outer cannula 502. Referring to FIG. 12, with the handle 542 of the stylet 506 positioned substantially perpendicular to the handle 504 of the outer cannula 502, the stylet 506 is loaded onto the outer cannula 502 by pushing the elongate rod 546 of the stylet 506 through the central passage 522 of the outer cannula 502 until the locking mechanism 540 extending from the handle 542 of the stylet 506 rests within the recessed region 530 of the handle 504. The locking mechanism 540 is a generally U-shaped member that includes lateral projections 547, 549 extending from either side. The lateral projections 547, 549 of the locking mechanism have contoured upper surfaces. After inserting the locking mechanism 540 into the recessed region 530 of the handle 504, the stylet 506 is rotated. This rotation causes the lateral projections 547, 549 of the locking mechanism 540 to slide under members 551, 553 (shown in FIG. 13) extending from sidewalls of the handle 504 into the recessed region 530. As the stylet 506 is rotated, the frictional resistance between the upper surfaces of the projections 547, 549 of the locking mechanism 540 and the lower surfaces of the members 551, 553 of the handle 504 increases due to the contoured shape of the upper surfaces of the projections 547, 549. The stylet 506 is rotated until the walls 543, 545 of the handle 542 make contact with side surfaces of the handle 504, as shown in FIG. 13. In this configuration, the stylet 506 is inhibited (e.g., substantially prevented) from moving axially relative to the outer cannula 502 due to contact between the projections 547, 549 and the members 551, 553. The walls 543, 545 also prevent the stylet 506 from being rotated in a clockwise direction relative to the outer cannula 502. The frictional contact between the upper contoured surfaces of the projections 547, 549 and the lower surfaces of the members 551, 553 can also help to prevent the stylet 506 from being rotated relative to the outer cannula 502.

Referring to FIGS. 11 and 13, the handles 504 and 542 are shaped so that the user can grasp the handles 504, 542 with the user's palm contacting the top surface of the handle 542 and the user's fingers wrapped around a bottom surface of the handle 504 when the stylet 506 is secured to the outer cannula 502. The tubular member 510 of the outer cannula 502 can be positioned between two adjacent fingers (e.g., between the middle finger and the ring finger) of the user when the handles 504, 542 are grasped in this manner. With the handles 504, 542 and the outer cannula 502 grasped in this manner, the user can apply an axial and/or rotational force to the tubular member 510 of the outer cannula 502 via the handles 504, 542 to help drive the tubular member 510 through bone and other tissue.

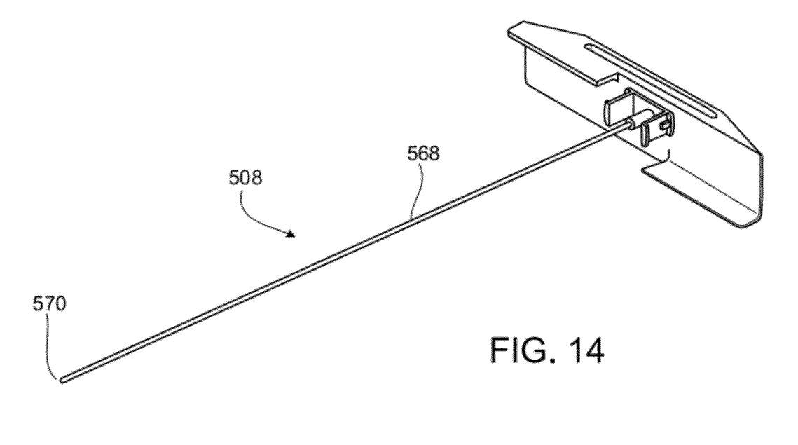

FIG. 14 shows a blunt-tipped stylet 508 that can be used interchangeably with the sharp-tipped stylet 506 in the bone cement delivery system 500. Unlike the sharp-tipped stylet 506, the blunt-tipped stylet 508 includes an elongate rod 568 with a blunt distal end 570. The blunt-tipped stylet 508 includes a handle that is substantially identical to the handle 542 of the sharp-tipped stylet 506. All other features (e.g., size, shape, materials, construction) of the blunt-tipped stylet 508 are also generally the same as the sharp-tipped stylet 506. The blunt-tipped stylet 506 can be secured to the outer cannula 502 using the securing technique described above with respect to the sharp-tipped stylet 506.

The bone cement delivery system 500 illustrated in FIGS. 10-14 can be used to perform any of the bone cement delivery methods described herein with respect to bone cement delivery system 100.

While the methods described above involve injecting bone cement paste into the femur through the central passage of the outer cannula 102, 502, other techniques can be used. In some embodiments, for example, after verifying the desired position of the bone cement delivery system 100, 500 within the femoral neck 306, the blunt-tipped stylet 108, 508 is removed from the central passage of the outer cannula 102, 502 and a longer (e.g., eight inch) blunt tip needle is inserted into the central passage. When fully inserted and restrained within the central passage, this needle protrudes beyond the distal end of the outer cannula 102, 502. A syringe filled with bone cement paste is then connected to the hub of the needle and the bone cement paste is delivered to the femoral neck 306 via the needle.

While the bone cement delivery systems above have been described as being used to inject osteoinductive bone cement paste into an unfractured femoral neck (e.g., to reduce the likelihood of a future fracture), the bone cement paste can also be injected into fracture sites within the femoral neck. In addition, the bone cement delivery systems above can be used to inject bone cement paste into other regions of the femur and into other bones.

While bone cement paste formed of injectable calcium phosphate matrix (CPM) and recombinant human Bone Morphogenetic Protein-2 (rhBMP-2) has been described, the bone cement delivery systems described herein can be used to inject any of various other types of bone cements. For example, while the matrix of the bone cement paste has been described as CPM, one or more other types of bone cement matrixes can alternatively or additionally be used. Examples include calcium phosphate based powders and polymethyl methacrylate based powders. Any of various osteoconductive powders, such as ceramics, calcium sulfate or calcium phosphate compounds, hydroxyapatite, deproteinized bone, corals, and certain polymers, can alternatively or additionally be used.

As an alternative to or in addition to using rhBMP-2, any of various other active agents can alternatively or additionally be used in the bone cement paste. The active agent of the bone cement paste can, for example, be selected from the family of proteins known as the transforming growth factor-beta (TGF-β) superfamily of proteins, which includes the actives, inhibits, and bone morphogenetic proteins (BMPs). In some embodiments, the active agent includes at least one protein selected from the subclass of proteins known generally as BMPs. BMPs have been shown to possess a wide range of growth and differentiation activities, including induction of the growth and differentiation of bone, connective, kidney, heart, and neuronal tissues. See, for example, descriptions of BMPs in the following publications: BMP-2, BMP-3, BMP-4, BMP-5, BMP-6, and BMP-7 (disclosed, for example, in U.S. Pat. Nos. 5,013,649 (BMP-2 and BMP-4); 5,116,738 (BMP-3); 5,106,748 (BMP-5); 5,187,076 (BMP-6); and 5,141,905 (BMP-7)); BMP-8 (disclosed in PCT WO 91/18098); BMP-9 (disclosed in PCT WO 93/00432); BMP-10 (disclosed in PCT WO 94/26893); BMP-11 (disclosed in PCT WO 94/26892); BMP-12 and BMP-13 (disclosed in PCT WO 95/16035); BMP-15 (disclosed in U.S. Pat. No. 5,635,372); BMP-16 (disclosed in U.S. Pat. No. 6,331,612); MP52/GDF-5 (disclosed in PCT WO 93/16099); and BMP-17 and BMP-18 (disclosed in U.S. Pat. No. 6,027,917). Other TGF-β proteins that may be useful as the active agent of the bone cement paste include Vgr-2 and any of the growth and differentiation factors (GDFs).

A subset of BMPs that may be used in certain embodiments includes BMP-2, BMP-4, BMP-5, BMP-6, BMP-7, BMP-8, BMP-9, BMP-10, BMP-11, BMP-12 and BMP-13. In some embodiments, the composition contains two or more active agents (e.g., BMP-2 and BMP-4). Other BMPs and TGF-β proteins may also be used.

The active agent may be recombinantly produced, or purified from another source. The active agent, if a TGF-β protein such as a BMP, or other dimeric protein, may be homodimeric, or may be heterodimeric with other BMPs (e.g., a heterodimer composed of one monomer each of BMP-2 and BMP-6) or with other members of the TGF-β superfamily, such as actives, inhibits and TGF-β (e.g., a heterodimer composed of one monomer each of a BMP and a related member of the TGF-β superfamily). Examples of such heterodimeric proteins are described, for example in published PCT Patent Application WO 93/09229.

U.S. Patent Application No. 61/160,063, filed Mar. 13, 2009 and entitled “Bone Cement Delivery Systems and Related Kits and Methods,” is incorporated by reference in its entirety herein.

Other embodiments are within the scope of the following claims.

What is claimed is:

1. A bone cement delivery method, comprising:
inserting a portion of a bone cement delivery device into a femoral neck of a femur of a patient; and
injecting bone cement paste into the femoral neck of the patient via the bone cement delivery system.

2. The bone cement delivery method of claim 1, wherein the portion of the bone cement delivery system is percutaneously inserted into the femoral neck of the patient.

3. The bone cement delivery method of claim 1, wherein the portion of the bone cement delivery device that is inserted into the femoral neck comprises a distal end region of a cannula.

4. The bone cement delivery method of claim 3, wherein the portion of the bone cement delivery device that is inserted into the femoral neck further comprises a distal end region of a stylet, and the stylet is positioned at least partially within a bore of the cannula.

5. The bone cement delivery method of claim 4, wherein a distal end of the stylet extends distally beyond a distal end of the cannula.

6. The bone cement delivery method of claim 5, wherein the distal end of the stylet is sharp.

7. The bone cement delivery method of claim 5, wherein the distal end of the stylet is blunt.

8. The bone cement delivery method of claim 4, wherein inserting the portion of the bone cement delivery device into the femoral neck comprises rotating the cannula and the stylet.

9. The bone cement delivery method of claim 8, wherein the stylet comprises a handle having at least one wall member configured to contact a handle of the cannula to substantially prevent the stylet from rotating relative to the cannula in at least a first rotational direction.

10. The bone cement delivery method of claim 2, wherein injecting the bone cement paste into the femoral neck comprises connecting a syringe to the cannula and operating the syringe to drive bone cement paste through a bore formed in the cannula.

11. The bone cement delivery method of claim 10, further comprising removing a stylet from the bore formed in the cannula prior to connecting the syringe to the cannula.

12. The bone cement delivery method of claim 1, wherein the bone cement delivery system comprises an outer cannula comprising an elongate tubular member defining a bore extending from a proximal end of the elongate tubular member to a distal end of the elongate tubular member, and a first stylet removably positioned at least partially within the bore of the elongate tubular member.

13. The bone cement delivery method of claim 12, wherein a distal end of the first stylet extends distally beyond a distal end of the cannula

14. The bone cement delivery method of claim 13, further comprising, after passing the distal end of the first stylet through a cortex of the femur of the patient, removing the first stylet from the bore and inserting a second stylet into the bore.

15. The bone cement delivery method of claim 14, wherein the distal end of the first stylet is sharp, and the distal end of the second stylet is blunt.

16. The bone cement delivery method of claim 12, further comprising fixing the first stylet relative to the outer cannula.

17. The bone cement delivery method of claim 16, wherein fixing the first stylet relative to the outer cannula comprises axially fixing the first stylet relative to the outer cannula.

18. The bone cement delivery method of claim 17, wherein fixing the first stylet relative to the outer cannula further comprises fixing the first stylet relative to the outer cannula in at least one rotational direction.

19. The bone cement delivery method of claim 16, wherein fixing the first stylet relative to the outer cannula comprises rotating the first stylet relative to the outer cannula to position a projection extending from the outer cannula within a slot defined by the first stylet.

20. The bone cement delivery method of claim 19, wherein the projection extends from a handle of the outer cannula, and the slot is defined by a head of the first stylet.

21. The bone cement delivery method of claim 16, wherein fixing the first stylet relative to the outer cannula comprises rotating the first stylet relative to the outer cannula to position projections of a locking member of the first stylet under members extending from a handle of the outer cannula.

22. The bone cement delivery method of claim 21, wherein the locking member is a U-shaped member extending from a handle of the first stylet, and the projections extend laterally from opposing sides of the U-shaped member.

23. The bone cement delivery method of claim 22, wherein the projections have contoured upper surfaces.

24. The bone cement delivery method of claim 12, wherein the elongate tubular member of the outer cannula has an outer diameter of about 0.094 inch to about 0.096 inch, and the elongate tubular member of the outer cannula has an inner diameter of about 0.075 inch to about 0.079 inch.

25. The bone cement delivery method of claim 12, wherein the elongate tubular member of the outer cannula has a length of about six inches.

26. The bone cement delivery method of claim 12, wherein the bone cement paste comprises calcium phosphate matrix (CPM) and recombinant human Bone Morphogenetic Protein-2 (rhBMP-2).

27. The bone cement delivery method of claim 12, wherein the bone cement paste is injected into a portion of the femoral neck that is not fractured.

28. The bone cement delivery method of claim 12, wherein the bone cement paste increases the bone mass of the femoral neck after a period of time.

29. The bone cement delivery method of claim 4, wherein inserting the portion of the bone cement delivery device into the femoral neck comprises striking the stylet with a mallet.

30. The bone cement delivery method of claim 4, wherein inserting the portion of the bone cement delivery device into the femoral neck comprises striking a strike plate of the stylet with a mallet.
